(19) United States
(12) Patent Application Publication
(10) Pub. No.: US 2011/0061649 A1
(43) Pub. Date: Mar. 17, 2011

(54) DEVICE AND METHOD FOR TREATING RESPIRATORY AND OTHER DISEASES

(76) Inventor: Israel Hirshberg, Alfei Menashe (IL)

(21) Appl. No.: 12/673,582

(22) PCT Filed: Aug. 17, 2008
(86) PCT No.: PCT/IL08/01129

§ 371 Date: Dec. 2, 2010

Related U.S. Application Data
(60) Provisional application No. 60/956,130, filed on Aug. 16, 2007.

(51) Int. Cl. A61M 15/00; A61K 31/105; A61P 31/04; A61P 31/12; A61P 31/10; A61P 33/00; A61P 31/16; A61P 11/06; A61P 11/04; A61P 11/00

(52) U.S. Cl. 128/203.12; 514/707

(57) ABSTRACT

A method and device to prevent and cure body diseases caused by infection factors such as: bacteria, virus, fungi, parasites as well as preventing fat accumulation in body blood vessels, lowering blood pressure, lowering cholesterol level in the blood and destroying body organs cancer cells is done by inhaling vapors of fresh crushed or sliced garlic or vapors of synthetic allicin.

### FIELD OF THE INVENTION The present invention relates to inhaler aimed to cure human diseases and more specifically to a method of treating respiratory and other diseases.

### BACKGROUND OF THE INVENTION

Infections of the respiratory tract are common in humans, especially in young infants and in the elderly are sometimes severe, causing complications and even death. These infections are usually treated by anti microbial agents, which are commonly associated with undesired adverse events, i.e. causing damage to human organs such as lever and kidneys. In addition, over the years, microorganisms causing respiratory infections—bacteria, viruses, parasites and fungi—have developed increasing resistance to the anti microbial agents, thus reducing their treatment efficacy and increasing the need and cost of advanced anti microbial agents.

Further, the current treatment of respiratory diseases caused by bacteria is by administering antibiotics drugs to the patient. Usually, these drugs are given through the patient's mouth or by injection. These drug administering methods bring the drugs into the patient's blood circulation system to the infected tissue. This method is not efficient in the case where infection factors reside in the air within the lung's bronchioles and alveoli. Since the white blood cells of the immune system and antibiotics in the blood stream cannot reach these air spaces. Thus, the patient is constantly attacked by bacteria and viruses that reside in this air spaces. This phenomenon lengthen the period of illness and force the patient to take more large doses of medicines, which could damage is kidneys and lever, as written in many of this medicines packages.

As a matter of fact there is an inhaler aimed to cure influenza (type A and B). This is the Zanamivir (also known as Relenza) inhaler. However, this drug found to be effective only when first influenza symptoms felt and the patient has no fever.

Therefore it is highly desirable to have an efficient method to treat wide range of infection factors attacking the respiratory tract without adverse side effects.

### SUMMARY OF THE INVENTION

According to the present invention there is provided a method of treatment human and mammals diseases caused by bacteria, virus, parasites, and fungi by inhaling crushed fresh garlic cloves vapors into human body or animal lungs. When fresh garlic cloves are crushed, allicin is generated within seconds in the crushed garlic. Allicin vapors naturally flow from the crushed garlic into surrounding air. Inhaling air reach with allicin vapors is a major aspect of the invention.

Allicin vapors inhaled into lungs' alveoli, pass alveoli membranes and enter into the blood stream within seconds after allicin is produced. Allicin carried by blood stream reach all body organs within seconds ready to destroy infection factors such as bacteria, virus, parasites and fungi.

Another aspect of the invention is generating allicin from its origin substances: alliin and enzyme alliinase and inhaling this allicin vapors.

Another aspect of the invention is inhaling allicin to fight body tumors.

Yet another aspect of the invention is inhaling allicin to prevent blood fat accumulation in body blood vessels.

Yet another aspect of the invention is inhaling allicin to lower platelet level in the blood system to prevent blood vessel clogging.

Yet another aspect of the invention is inhaling allicin to lower sugar level in the blood system.

Yet another aspect of the invention is inhaling allicin to lower body blood pressure.

Yet another aspect of the invention is to lower cholesterol level in the blood.

Yet another aspect of the invention is to fight cancer cells in the entire body

Yet another aspect of the invention is the use of a device that enables air flowing toward body mouth or nose to absorb natural crushed garlic vapors or synthetic allicin vapors to be inhaled.

Yet another aspect of the invention is the prevention of solid garlic particles entering the respiratory system by filtering the stream of inhaled air.

Yet another aspect of the invention is the prevention of solid garlic particles entering the respiratory system by letting the airflow carrying allicin to flow through small opening into wide pipe thus slowing the airflow which allows heavy than air particles to fall down and not taken into respiratory system.

Yet another aspect of the invention is a inhaler in which fresh crushed garlic is placed in it, thus the inhaled air flows through this inhaler to absorb garlic vapors and carry them into body respiratory system.

Yet another aspect of the invention is the incorporation of a powered fan that causes air to flow through a inhaler, thus carry allicin into patient who cannot breath.

Yet another aspect of the invention is a non personal fan powered allicin (garlic vapors) inhaler that feels room air to treat more than one human or animal residing in this room.

Yet another aspect of the invention is that a patient holds his breath while his lungs are full with garlic (allicin) vapors, to prolong allicin activity against infection factors in the small lung air spaces as bronchioles and alveoli.

Yet another aspect of the invention is of using other vegetables that produces strong scent when crushed such as: onions, horseradish and radish.

Still another aspect of the invention is the use of fresh crushed garlic vapors or synthetic allicin against important and virulent bacteria pathogens to humans, such as Staphylococcus aureus, Streptococci, pathogenic Escherichia coli, Proteus spp, Psudomonas and Acinetobacter.

Yet another aspect of the invention is to use inhaled allicin against virulent viruses such as: Herpes family, Influenza viruses, CMV, Parainfluenza and many others.

Yet another aspect of the invention is to use inhaled allicin against parasites including Amoeba, Giardia and Leishmania species.

Yet another aspect of the invention is to use inhaled allicin against fungi including Candida albicans, Aspergillus spp, which are of prime importance in immunocompromized patients, whose numbers are constantly increasing in modem medicine.

Yet another aspect of the invention is a device that produces allicin from its origin materials and form jet of allicin droplets and vapors into patient mouth.

Yet another aspect of the invention is that the source of garlic scent is by chewing a fresh garlic clove, leave it in the mouth for few seconds so and then carefully inhale slowly through the mouth and holding the intake air with garlic scent for about 10 seconds or more.

Still another aspect of the invention is that a source of garlic scent is by crashing few fresh garlic cloves and keeping the crushed garlic in perforated container close to the patient mouth or between his lips, thus when the patient breaths, the air coming to his lungs contains garlic scent.

Still another aspect of the invention is taking a session of inhaling garlic scent, i.e., several garlic inhaling breaths follow one another and then breathing fresh air.

Still another aspect of the invention is that a patient should take at least one garlic scent inhaling session per day.

Yet another aspect of the invention is that an entire population under risk of pandemic of influenza, SARS, or bird flue, inhale garlic vapors, i.e. allicin, simultaneously to destroy all infectious organisms in the entire population.

Still another aspect of the invention is that patient of lung disease and people under risk chew raw garlic cloves and swallow their mouth juice which has concentrated garlic juice that directly come in contact with the mouth and throat tissues to directly attack infected tissues.

Still another aspect of the invention is a garlic scent inhaler provided with at least one air filter to prevent garlic particles to flow into the patient lungs.

Still another aspect of the invention is a garlic scent inhaler provided with means to crash garlic cloves.

Yet another aspect of the invention is an inhaler made of several components so it can be dismantled for easy cleaning.

### BRIEF DESCRIPTION OF THE DRAWINGS

The present invention will be further understood and appreciated from the following detailed description taken in conjunction with the drawings which:

FIG. 1 is a cross section view through one embodiment of the invention using fresh crushed garlic or another vegetable.

FIG. 2 is another embodiment of the invention where the inhaler is equipped with garlic clove crasher.

FIG. 3 is another embodiment of the invention showing an inhaler equipped with electric powered fan.

FIG. 4 is a cross section of another garlic inhaler embodiment.

FIG. 5 is a cross section of another inhaler according to the invention having a vertical part.

FIG. 6 is a safety addendum part to be added to the embodiments of FIGS. 4 and 5.

FIG. 7 is another embodiments of the invention that uses allicin liquid container.

FIG. 8 is another embodiments of the invention that use allicin origin ingredients.

FIG. 9 is another embodiment of the invention having an allicin producer and electrical fan.

### DETAILED DESCRIPTION OF THE INVENTION

The present invention discloses a method of treatment respiratory and other infections by inhaling vapors produced when vegetables are crushed. Moreover, active ingredients in edible vegetables vapors can be inhaled into the lungs of humans and live stock and to enter the blood stream to effectively reach any organ and to fight infections or provide other therapeutic influence. It was proved laboratory tests that when fresh garlic (Allium sativum) cloves are crushed a new substance, named allicin is produced. Allicin has been studied and found to be effective against many species of bacteria and viruses. However, those scientific papers didn't mention any method of administering allicin to human body. Further, allicin have short life span of less than an our in room temperature.

Moreover, allicin was suspected to be poisonous and medical doctors, in general, disapprove inhaling of garlic vapors.

Although garlic is known as a food that helps in curing cold for thousands of years, no one before suggested a safe inhaler that produces garlic vapors so they can easily and safely inhaled. Some garlic experts as Eric Block, Ph.D. even spoke against eating raw garlic cloves since they are “very irritating and could injure the digestive tract”—see www.healthy.net “The Chemistry Of Garlic Health Benefits”.

However, the inventor decided to examine inhaling garlic vapors on himself. He found that breathing deep breaths of garlic vapors and holding the breath for 20 seconds or so has high heeling effect against wide range of respiratory diseases and soar throat. In case of soar throat, which could lead to high fever, inhaling garlic vapors lowered the throat pain level with 30 minutes and prevented high body temperature. These two symptoms proved the high efficacy of inhaling fresh crushed garlic cloves scent, i.e. vapors. Later he found that fresh garlic vapors contain the well-known allicin.

Since allicin proved to be highly vaporized it can easily mixed with inhaled air and reach the lung's bronchioles and alveoli and there to pass the membrane into the bloodstream which takes the allicin to every organ in the entire body.

In an article published by Weizman Institute Research in Israel on Oct. 14, 1997 it says: “One study, appearing in the October issue of the American Society for Microbiology's Antimicrobial Agents and Chemotherapy, explains how allicin fights infection. This research supports the notion that garlic is an excellent, although smelly, natural antimicrobial drug that can disable an unusually wide variety of infectious organisms.”

This article further says; “The second study, soon to be reported in Biochimica Biophysica Acta, may help clarify the role allicin plays in preventing heart disease and other disorders.

### Natural Weapon Against Infection

The research reported in October's Antimicrobial Agents and Chemotherapy revealed that allicin disables dysentery-causing amoebas by blocking two groups of enzymes, cysteine proteinases and alcohol dehydrogenases.

Cysteine proteinase enzymes are among the main culprits in infection, providing infectious organisms with the means to damage and invade tissues. Alcohol dehydrogenase enzymes play a major role in these harmful organisms' metabolism and survival.

Because these groups of enzymes are found in a wide variety of infectious organisms such as bacteria, fungi and viruses, this research provides a scientific basis for the notion that allicin is a broad-spectrum antimicrobial drug, capable of warding off different types of infections.

“It has long been argued that garlic can fight a wide range of infections, and now we have provided biochemical evidence for this claim,” says Prof. Mirelman.

The role of allicin in warding off infection may be particularly valuable in light of the growing bacterial resistance to antibiotics. It is unlikely that bacteria would develop resistance to allicin because this would require modifying the very enzymes that make their activity possible.

### Blocking Mechanism Explained

In the study slated to appear in Biochimica Biophysica Acta, Institute scientists found that allicin blocks the enzymes by reacting with one of their important components known as sulfhydryl (SH) groups, or thiols.

This finding has important implications because sulfhydryl groups are also crucial components of some enzymes that participate in the synthesis of cholesterol. By reacting with and modifying the sulfhydryl groups in those enzymes, allicin may prevent the production of artery-clogging cholesterol.

“It has been suggested that garlic lowers the levels of harmful cholesterol, and our study provides a possible explanation for how this may occur,” says Prof. Wilchek. “However, more research is necessary to establish what role allicin might play in preventing the clogging up of arteries.”

Complicating the issue is the concern that blocking sulfhydryl groups in proteins may sometimes be harmful because these groups are also present in enzymes involved in some of the body's vital processes. However, unlike most bacteria, human tissue cells contain detoxifying molecules of a substance called glutathione, which helps maintain appropriate sulfhydryl levels. These glutathione molecules can reverse the anti-sulfhydryl effects of small amounts of allicin.

### Measuring Antioxidant Activity

While reaction with sulfhydryl groups appears to explain most of allicin's activity, it has also been suggested that allicin acts as an antioxidant. The study reported in BBA confirmed this antioxidant effect and for the first time provided its quantitative assessment. Antioxidants gobble up harmful free radicals believed to contribute to tumor growth, atherosclerosis, aging and other processes.

### Producing Pure Allicin in Large Quantities

In nature, allicin is created when garlic cloves are cut into or crushed. The cutting or crushing causes two components of garlic, alliin and the enzyme alliinase, to interact.

The allicin produced at the Weizmann Institute is semi-synthetic; first, its precursor, alliin, is chemically synthesized, then a modified form of the natural enzyme, alliinase, converts it into pure allicin.

The pure semi-synthetic allicin can be stored for months without losing its effectiveness. In contrast, the natural compound loses its beneficial properties within hours because it begins to react with garlic's other components as soon as the clove is crushed.

A patent application for this production of pure allicin has been submitted by Yeda Research and Development Co., the Institute's technology transfer arm, and several companies have already expressed interest in scaling up the process for commercial use and clinical testing.

Prof. Mirelman, the Weizmann Institute's Vice President for Technology Transfer, holds the Besen-Brender Chair of Microbiology and Parasitology, and Prof. Wilchek, Dean of the Biochemistry Faculty, holds the Marc R. Gutwirth Chair of Molecular Biology.

This research was funded in part by the Center for Molecular Biology of Tropical Diseases at the Weizmann Institute and the Avicenne Program of the European Union. Drs. Rabinkov and Konstantinovski were partly supported by the Center for the Absorption of Scientists of Israel's Ministry of Absorption.

The Weizmann Institute of Science is a major center of scientific research and graduate study located in Rehovot, Israel.

“The studies were led by Profs. David Mirelman and Meir Wilchek of the Institute's Biological Chemistry Department, who worked together with departmental colleagues Drs. Serge Ankri, Talia Miron and Aharon Rabinkov and with Prof. Lev Weiner and Dr. Leonid Konstantinovski of the Organic Chemistry Department.”

Allicin proved to be highly effective against viruses.

In an article by Susumi Yoshida and others: “Antifungal activity of Ajoene Derived from Garlic”, published in “Applied and Environmental Microbiology” March 1987 P 615-616, the authors describe the anti fungal activity of Allicin.

Another Garlic expert, Mr. Ronal Cutler, a senior lecturer in microbiology in the University of East London, formulated allicin into skin products such as creams and soap and achieved success in destroying the microbes in laboratory tests. See www.rense.com. In this article Mr. Cutler reports that allicin “blast worst antibiotic resistant bacteria”: “killing of methicillin-resistant staphylococcus aureus” and vancomycin-resistant enterococci, an intestinal bacteria that causes considerable illness and death in hospital settings.”

In the recent years, several scientific papers have shown that Allicin, one of the garlic principle is highly active in destroying viruses, bacteria and parasites, fungus as well as possessing anti-tumor activity, lowering fat levels and sugar levels in human blood, lowering blood pressure, inhibit platelet functions and prevents Chronic Pulmonary Hypertension. On the other hand, mammals cells proved to be immune to medium levels (50 Mm) of see article: exhausted “Allicin from Garlic Strongly Inhibits Cysteine Proteina and Cytopathic effects of Entamoeba hystolytica” by David Mirelman, Weizmann Institute of Science, Israel, published in Anti microbial Agents and Chemotherapy, October 1997, p 2285-2288. Another article “Inhibition of tumor growth by novel approach: In situ allicin generation using targeted alliinase delivery”, by Talia M iron at al published in Molecular Cancer Therapeutics 1295.

Limited established treatment options exist for the treatment of serious, invasive infections caused by multidrug-resistant Staphylococcus aureus, most notably nosocomially acquired methicillin-resistant S. aureus (MRSA). This MRSA exist in hospitals and it became a serious threat since no known drug proved to be effective. I is believed that Allicin could be effective against MRSA since it was not exposed to Allicin and not developed resistant to Allicin.

Allicin proved to be very useful in treating humans various health problems however, it also proved to be unstable compound having life span of about an hour in room temperature. This explains why allicin didn't turn to be widely used as commercial product. Farther, understanding allicin short life explains why allicin created in a human mouth when chewing fresh garlic cloves and then swallowed, is highly ineffective method of enjoying allicin benefits. Since it takes several hours from allicin creation in the mouth until it reaches the small intestine where it can enter the blood stream, by then, most of the allicin already transformed into other compound—not effective as allicin—especially in the stomach, where it is attacked by acids and enzymes. Thus swallowing allicin is poor way to put allicin into action.

However, inhaling fresh garlic vapors introduces Allicin into human respiratory tract and lungs within seconds from its creation. Allicin proved to be able to pass human membranes thus leaving the lungs alveoli and entering the blood stream and then entering human cells to act against virus. Thus inhaled allicin is active for period of hour, fighting infections and tumors all over the body and in every organ since the blood stream carry this Allicin from the lungs to the entire body. Therefore inhaling Allicin is the best way of using Allicin as drug to treat all mentioned health problems.

When fresh garlic cloves are chewed they produce very hot juice that many humans cannot stand, however inhaling fresh garlic scent is not that irritating thus it is possible for healthy and ill people to inhale garlic scent several times a day which is necessary in preventing and healing influenza and other diseases.

The devices disclosed in this application overcome the short life span of Allicin and its tendency to react with other garlic compounds or human acid and enzymes in the stomach that eliminate Allicin molecules.

It seems that lung diseases start at human mouth and throat as local infection. The infecting bacteria or virus, cling to the mouth and throat tissues where they find good conditions to multiply. Later, these infection factors are carried by inhaled air toward the lungs to start new colonies in the alveoli. From there the infection factors penetrate the alveoli walls to enter the blood stream. When this happen, the human is under serious infection attack.

The colonies in the alveoli are quite protected from white blood cells that cannot attack virus and bacteria leaving on the alveoli walls and in the air. Thus infected human, or animal, immune system or drugs cannot attack these colonies, which are beyond reach. The existence of these colonies in the moist air within the alveoli prolong the time needed for a human to heal. This situation calls for an aerial attack, i.e., to inhale air which includes therapeutic agent, i.e. allicin droplets that enters the alveoli and kill the bacteria and virus that live in the air. This efficient aerial attack explains the high efficacy of inhaling allicin which kill the infection factors hiding from white blood cells. It was proved that inhaling fresh garlic scent could prevent and cure influenza as well as keeping body temperature at healthy level by eliminating infection factors.

One option of creating allicin is by chewing a piece of fresh garlic clove for about a minute, leave the chewed garlic in the mouth for preferably another minute and while the mouth is full with garlic scent, slowly and carefully breath through the mouth so the intake air is absorbing the garlic scent and carry it into the lungs. Now this inhaled air is preferably trapped in the lungs by not exhaling the air for 20 to 30 seconds. This inhaling and trapping the air with garlic scent is preferably done for several times, to be referred here as a “inhaling session”. From the inventor own experience, repeating the garlic inhaling session for several times during the day has highly curing effect. As a matter of fact, this method have proved to be so efficient that implementing it in the early stage of the infection may cure the infected person within two days and preventing the person to face severe lung disease and high fever. However, this method could be dangerous if the person unintentionally intake a piece of garlic into his lungs. To prevent this dangerous case, the inhalers of this application were invented.

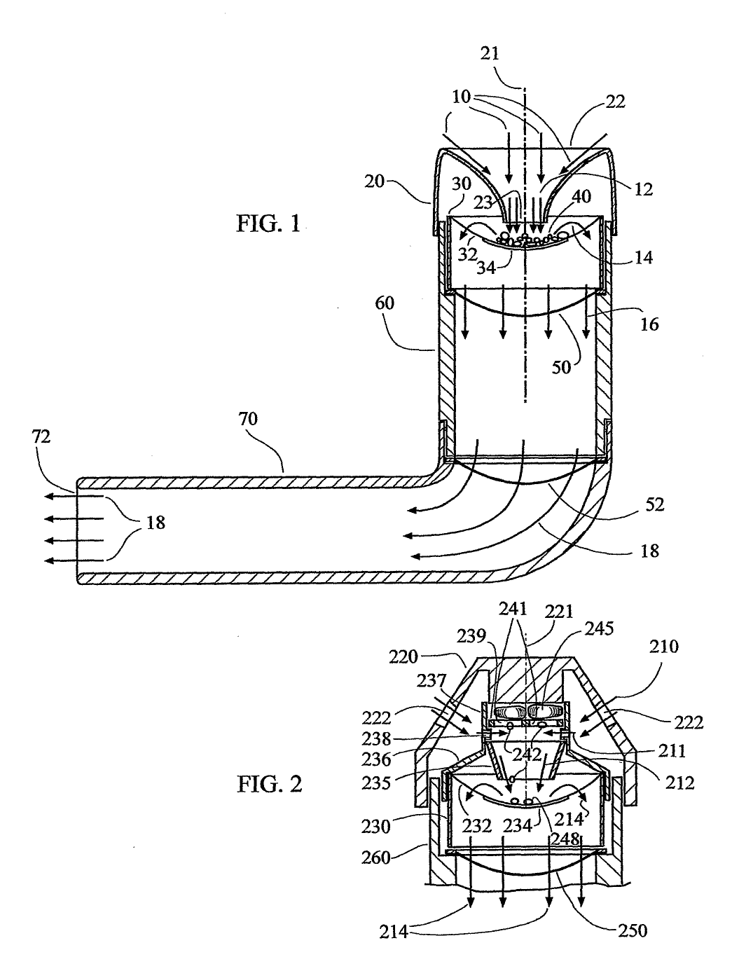

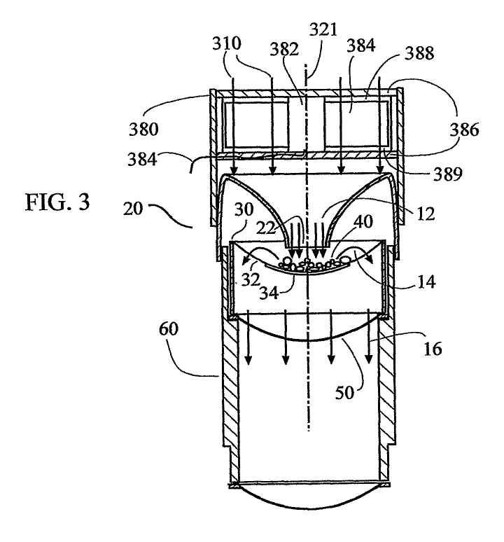

Further, most of western countries population cannot stand the hot taste of crushed garlic cloves in their mouth. For these people, the garlic scent inhaler is good solution. The same is with children. Some patients having pneumonia such babies and elderly cannot chew garlic cloves and may suffer breathing problems. For this population the powered inhaler was invented—see FIGS. 1 and 3. This device generate a stream of air enriched with allicin vapors. The air stream should be directed toward the mouth or nose of the patient, if he/she can breath autonomy. In case these patient needed to breath with the help of breathing machine, this inhaler could provide air with allicin to the breathing machine.

The fresh garlic scent inhalation was tested by the inventor on itself and proved highly efficient in curing influenza, cough and soar throat. However the scientific studies made on allicin proved it is highly effective against many kinds of infection virus and bacteria and this give reason to believe allicin can cure bird flu and SARS.

This invention has the advantage of curing and preventing the lung disease by small investment of few garlic cloves per person. Thus an entire population can chew garlic and inhale the garlic scent or use the innovative inhalers described in this application for several days until all the infected persons can be cured at the same time and there will be no human being to inhabit the cause of the disease, thus eliminating it at country or global scale.

The perform large scale population immunization the state authorities should explain the method of inhaling garlic scent as described above on TV channels and on radio stations and ask the entire population to repeat the inhaling session for several times per day for a duration of several days.

Garlic has been eaten for thousands of years. When eating garlic human breath is vapors, i.e. allicin, thus humanity has been exposed to allicin. This exposure didn't create adverse side effect that could occur when using antibiotics. The lack of adverse side effects of allicin is important for fast and short and low cost regulation that might be required to allow marketing of garlic inhalers.

One major aspect of this invention is the inhaling of vapors produced by crushing fresh garlic cloves. Other aspects of the invention is that the crushing is done within the inhaler and by the inhaler.

FIG. 1 is a cross section view of a garlic scent inhaler according to one embodiment of the invention. The inhaler helps people who cannot stand the presence of garlic in their mouth to breath garlic scent in a safe and comfortable manner.

The device comprises of a pipe 70 preferably cylindrical, made of solid or semi solid material, preferably high quality plastic, which can be sterilized—through which the patient breathes. The pipe end 72 is placed in the patient mouth. The arrows 18 symbolize the scented air, flows into the patient mouth by his lungs suction power. The device other end 20 has an inlet 22, where a convergent nozzle starts and ends at section 23. This nozzle accelerates the inhaled air 10 to form air jet 12, which hits fresh crushed garlic particles 40. The garlic is preferable fresh crashed garlic or fresh sliced garlic, which emit garlic vapors i.e., allicin droplets into the air jet 12. The garlic particles are placed in a bowl 34, made of solid or elastic material preferably plastic, metal, glass or alike. The bowl is attached to porous membrane 32, which allows the scented air 14 to pass through it down as scented airflow 16. The porous membrane 32 serves as a first filter to prevent garlic particles to be inhaled into the patient lungs. The membrane and bowl are attached to a cylindrical pipe 30 which is fitted into cylindrical pipe 60 top opening and can be easily dismantled from it for cleaning purposes. As can be seen, the inlet outer body 20 is attached to pipe 60 external surface by threads or just tight fit. At the bottom of pipe 30 an optional air filter 50 is located, where its rim sits in pipe 60. Optionally filter 50 can be dismantled for cleaning purposes. The air filter 50 holes are adapted to prevent small garlic particles that have been swept with the flow 14 to be trapped at the air filter 50. The vertical pipe 60 is connected to the pipe 70 thus the scented air 18 leaves the pipe 60 into pipe 70 toward the patient mouth. An optional air filter 52 is placed to further filter the scented air from any particles remained. It should be noted that the membrane area 32 through which scented airflow 14 passes is greater than the cross section 23 thus decelerating the air speed and decreasing the aerodynamic force on the garlic particles to be pushed through porous membrane 32, thus these garlic particles that already swept to the membrane cannot pass it and fall back into the bowl when the patient stops inhaling

FIG. 2 shows optional improvement the device of FIG. 1. Here, garlic cloves crasher 220 is provided to eliminate the need of crushing the garlic cloves outside of the inhaler. On top of cylinder 230 (identical to element 30 of FIG. 1) a cylindrical hollow tower 236 is placed. On top of tower 236 a rigid disk 239 has plurality of holes 241, preferably made of metal or tough plastic, is placed. On the top of disk 239 two garlic cloves 245 are placed. A rigid cylindrical element 220 is mounted on pipe 260 (pipe 260 corresponds to element 60 of FIG. 1) preferably by threads. Thus, when element 220 is twisted clockwise, it engages in the threads of element 260 and forced to go down to exert force on the garlic cloves, which are pushed toward the disk 239. While the element 220 is further screwed on pipe 260, the applied force crushes the garlic cloves and garlic particles 242 fall through holes 241 into the bowl 234 and accumulate there. The element 220 has several holes 222 through which inhaled air 210 is entered and flows through passages 238 as airflow 211, redirected by a convergent nozzle 235 downward toward the garlic particles 248 in the bowl 234. The convergent nozzle 235 also accelerates the flow 211 to form a jet aimed at the garlic particles placed on the bowl 234.

FIG. 3 shows another embodiment of the garlic inhaler. This embodiment optionally replaces placed on the top of the inhaler of FIG. 1. This embodiment comprises of an electrical powered fan 380 (similar to PC case ventilation fan), which is mounted on top of inlet 20 of FIG. 1 or on top of element 220 of FIG. 2. The fan hub 382 contains a small DC motor, similar to that found in personal computers. The fan blades 384 rotate and force air 310 to flow downward into the convergent inlet where the airflow 310 turns into air jet 12. This addendum should help patients who have difficulty to breath by themselves. The electrical power for the DC motor could be a battery or DC power supply.

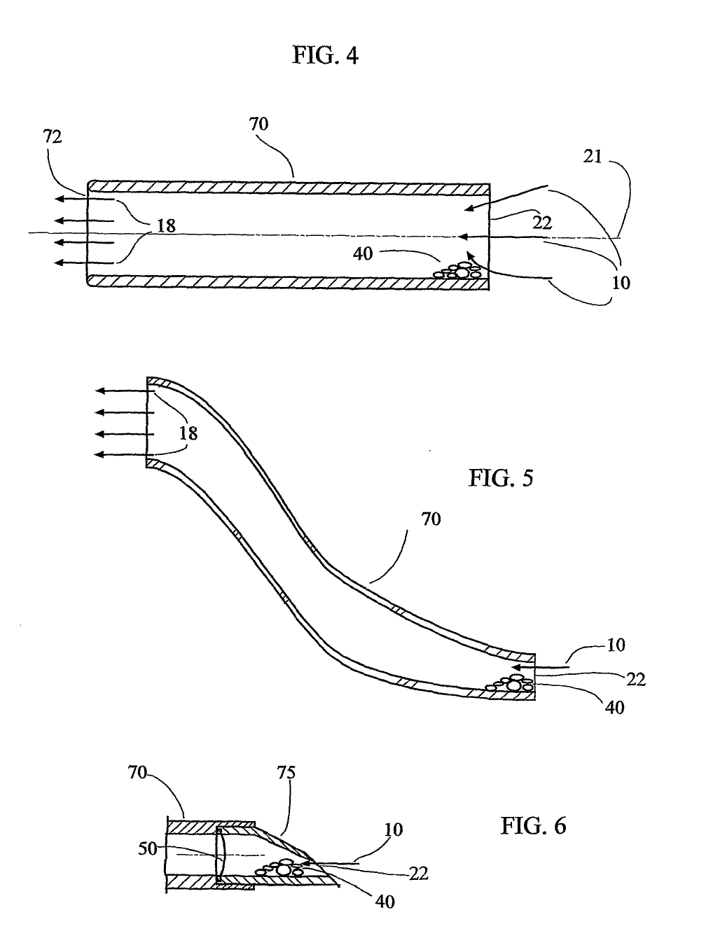

FIG. 4 shows another embodiment of the invention. A simple device comprises of a pipe 70 serves to hold the garlic particles far from the patient mouth. The patient breathes slowly, thus the garlic particles 40 are not swept with the incoming flow 10, which passes on the garlic particles and absorbs garlic scent and vapors.

FIG. 5 shows another embodiment of the invention where a vertical bent pipe 70 serves as an inhaler, The vertical part serves as bather to garlic particle to be inhaled. The flow 18 is entered the patient mouth while the coming fresh air 10 enters the pipe through inlet 22 where it hits garlic particles 40, absorbs their scent, i.e., allicin and flow into the patient mouth while he breathes. To prevent garlic particles to be swept into the patient mouth, the pipe 70 is curved into steep pipe in the side of the patient mouth so that gravity will pull garlic particles down and away from the patient mouth. Moreover, the intake area 40 is smaller than most of the pipe 70 cross sections, thus entering airflow 10 is faster than the flow in the rest of the pipe. This helps the airflow 10 to sweep garlic vapors from the pile of crushed garlic particles 40 and then to slow down to prevent garlic particles to enter the patient mouth.

FIG. 6 shows an addendum to the devices of FIGS. 4 and 5. A divergent nozzle 75 has small entrance 22 for fresh air 10 where particles of garlic 40 are placed. After the air enters the pipe 75 it flows into bigger cross sections and decelerates thus letting garlic particles to land before the scented air passes through the air-filter 50 which further traps small airborne garlic particles. Adding this device to the device of FIGS. 4 and 5 adds to the safety of using these inhalers.

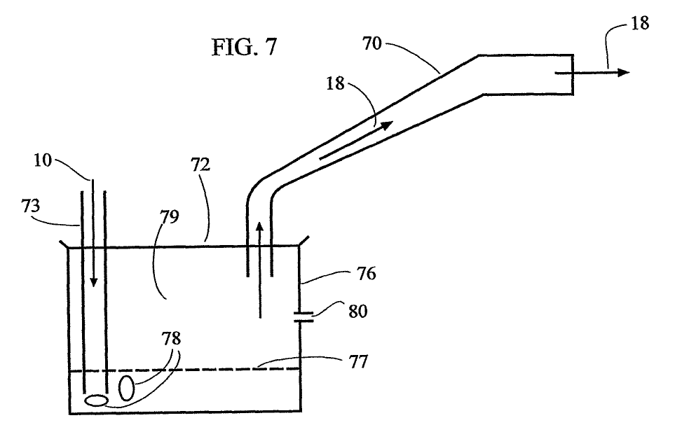

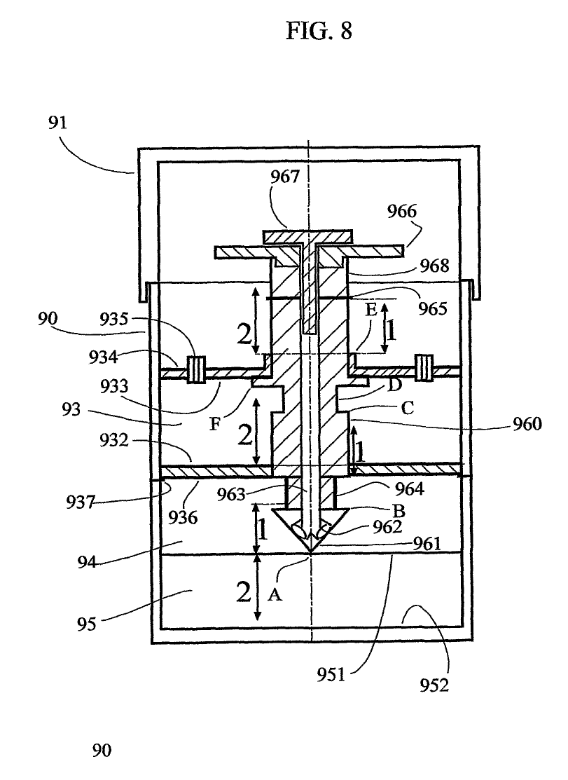

Allicin can be preserved stable for months at temperature of 4° Celsius. When allicin will be available as commercial product it can be used in a device show in FIGS. 7 and 8. These devices can be used for synthetic allicin as well. These devices extract Allicin or Allicin vapors from the allicin liquid and the patient inhale air with allicin vapors in deep breaths so the vapors reach the patient lungs and penetrates the human cells membrane to dissolve in the blood stream. It highly recommended that allicin be warmed to room temperature to accelerate its evaporation so that the quantity of vapors in each breath is at minimum level to become effective.

FIG. 7 shows another embodiment of the invention. Here, instead of crushed garlic, allicin extracted from garlic or synthetic garlic is used. A closed container 76 is partly filled with liquid allicin 77. The patient put the right side of pipe 70 in his mouth and breath. The air taken from air space 79, flows into the patient lungs and forms low pressure in the air space 79. Due to the low pressure in air-space 79, external air 10 is sucked through pipe 73 into the device and flows into the allicin liquid in the shape of bubbles 78. As the bubbles travels in the allicin liquid, up to the surface to fill the space 79, they absorb allicin vapors from the liquid, so the space 79 contains air and allicin vapors. Thus the next breaths will be rich with allicin vapors. To increase the amount of allicin vapors in the air-space 79, the device may by shaken for few seconds. An optional safety opening 80 prevents over filling of the device with liquid so that the bottom opening of pipe 70 is kept in air-space 79. The container 76 optionally has a removable lid 72.

Referring to the embodiments of FIG. 7 the container 76 is kept in refrigeration to prevent allicin disintegration while its openings are sealed by easily removable parts like plastics, rubber etc. Before the content is being used, it is preferably warmed to room temperature to increase the vaporization process, then the necessary tubes 73 and 70 are put to the described position and the patient may inhale through the pipe 70 allicin vapors.

It should be noted that existing devices such deodorant sprayer can be used to contain liquid allicin under pressure of inert gases, which do not chemically react with allicin. Such containers should be kept in refrigeration to prevent allicin from disintegration. When a patient needs to inhale allicin vapors, he sprays a jet of allicin droplets into his mouth and inhales air. The airflow evaporates the allicin, which its vapors are carried into the patient lungs. Also, containers, which contain the origin compounds of allicin: allicin and enzyme alliinase, separated is disclosed in FIG. 8. When a patient needs to inhale allicin vapors, he causes the two ingredients to mix and then shakes the container to accelerate the formation of allicin and then spray a jet of allicin droplets into his mouth. After spraying allicin droplets in his mouth the patient breath thus transferring allicin vapors into his lungs. The importance of such device is that allicin origin ingredients are stable compound while allicin life span is few hours. Therefore, such a device enables synthetic production of allicin ingredients and keeping a ready to use allicin inhaler without refrigeration.

FIG. 8 is a container of allicin origin ingredients. A cylindrical container 90 is separated into four compartments. Compartment 93 is slightly pressurized over 1 bar containing a gas like nitrogen or co2, i.e. inert gas. Compartment 94 contains alliinase and compartment 95 contains alliin. A thin membrane 951 separates the two ingredients which each can be encapsulated in its own container before assembly in container 90. A pin 960 equipped with hard arrow shape end 961 has inner channel 963. A seal 967 seals the channel 963 top end. To use this allicin inhaler, the cap 91 is removed to allow human access to the disc 966. The user pushes pin 960 head 967-966 down until he fills some resistance due to thin disk 965 is in contact with surface “E” which is part of element 934. At this point the pin 960 already broke membrane 951 so that the two ingredients in compartment 94 and 95 now intermixed to generate allicin. The same process takes place when fresh garlic clove is crushed and the two ingredients within the clove intermixed to create allicin. To accelerated the allicin generation, the container 90 may be shaken by hand for about 2 to 30 seconds and then the seal 967 is removed and the pin 960 is further pressed so that the center hole of wall 932 that was sealed by pin 960 is now open since zone “D” which its diameter is smaller than the hole diameter in wall 932, so that when zone “D” is positioned in the hole, a passage is formed between chamber 93 and chamber 94 so pressurized gas from chamber 93 passes through the hole in wall 932 to push allicin in compartments 94,95 through holes 962 up in channel 963 and outward into the patient mouth.

The pressurized compartment is one option to push the allicin up through channel 963. Another option is to install at least three supporting pins 937 so they form a plane parallel to bottom surface 952 and support disk 932. Disc 932 lies on these pins, which will be sheared by slight force exerted by surface “F” on disk 932. Firstly, the user remove the cup 91, Secondly he presses the pin head 966-967 downward so that the pin sharp edge 961 breaks the wall 951 and the origin compounds intermixed to create allicin. Thirdly, he shakes the container for few seconds and fourthly removes the seal 967. Fifthly, he further pushes the pin heads 966 downward so that surface “F” pushes disk 932 and breaks the supporting pins 937. Then, further pressing the pin 960 downward pushes the disk 932 downward, which press the allicin to flow upward through holes 962 and channel 963 into the patient mouth. Now the patient inhale and the airflow evaporates the allicin in his mouth and the air mixed with allicin inhaled into the patient lungs.

To assemble this inhaler, the following steps should be taken: first container 95 is placed in the bottom of container 90. This sealed container contains one allicin ingredient compound. Then container 94 is placed on top of container 95. Container 94 is empty in this stage and has a circular hole in its top disk 932 to accommodate pin 960 that will sill the hole in disk 932. Then pin 960 is inserted through the hole in disk 932. Then disk 934 is inserted on the pin 960. Disk 934 is pressed into the container to slide on pin 960 until encounter the disc “F”. While disc 934 is in its place, it is tightly fit in its place and the friction force between disk 934 and the walls of container 90 is high to prevent the disc to move upward due to the pressurized gas to be put in compartment 93. Optionally disc 934 may be glued or plastic welded to container 90 walls, Now, a measured amount of the other ingredient of allicin is injected through channel 963 into compartment 94. Then, the channel 963 is sealed by a seal 967. Now, few droplets of liquid nitrogen are injected through valve 935. This can be done by a syringe or similar device. The liquid nitrogen is at low temperature so that after few seconds the nitrogen absorb heat from the container and vaporize to form pressurized gas within chamber 93.

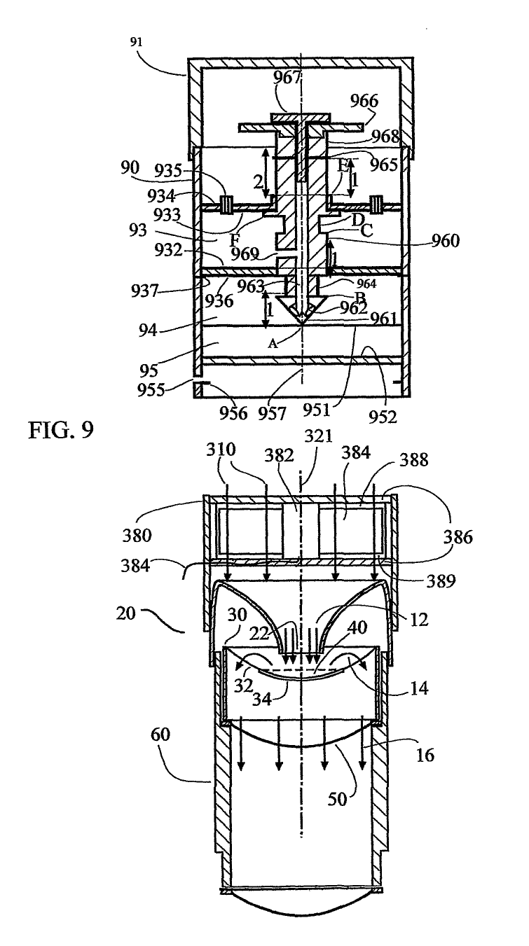

FIG. 9 discloses another embodiment of the invention, where the embodiment of FIG. 8 is combined with the embodiment of FIG. 3. Similar combination can be done with the embodiment of FIG. 1. These combinations form inhaler that uses allicin and mixing it with air to be inhaled by a patient. The allicin container 90 of FIG. 8 requires has some changes. They are:
1) There is new hole 969 in pin 960 that allows allicin flow from compartment 94 through channel 963 downward through holes 962—after the pin 960 head breaks the bottom wall 952, so that allicin is sprayed into the intake of the electric fan 384.
2) Decreasing the height of compartment 95 so that pin 960 in its second move breaks the bottom 952 and allicin flows through holes 962 and sucked by fan 384.
3) Air holes 955 through which air is sucked by the fan downward and intermixed with the allicin ejecting from pin 960.
4) Elongated container 90 walls downward to form a cylindrical space 957 into which the cylinder 380 enters.
The inhaler of FIG. 3 has one change which is the bowl 34 now adapted to hold liquid allicin.
The devices shown are separated to demonstrate how the two parts are shown before they are combined to be used. Before use, the one time use container 90 is installed on top of the electrical powered embodiment of FIG. 3. Then, a DC battery operates the electrical-fan 384. Then cap 91 is removed, the user performs two steps: 1), pressing pin 960 downward to form allicin (as explained for FIG. 8) and to eject it to the fan 384 inlet. The allicin is gathered in bowl 34 where airflow 310 hit the allicin an evaporate it, thus allicin vapors flow in airflow 14, 16 into a space or human mouth.

It should be noted that the embodiments described in this document are for personal use consequently their sizes are adapted to be hand held. However, the embodiment of FIG. 3 can be used as large device for group therapy.

The invention could be used for a group of human patients residing in a room into which, garlic scented air—air with allicin—is flow and the patients in the room breathe it. Many different system can be designed for this purpose all based on the principles described before where airflow driven by mechanical mean like fan or other mean, hits garlic, absorbs its scents and vapors and flow into the room to be breath by the patients. This method can be applied to poultry to treat bird flu so they can be cured instead of destroy.

It should be noted that garlic cloves might be replaced by other strong scent producing vegetables especially those who produces their scent when crushed such as: horseradish, radish, onion etc'.

It will be appreciated that the invention is not limited to what has been described hereinabove merely by way of example. Rather, the invention is limited solely by the claims, which follow.

1. A method of preventing and curing body diseases caused by infection factors such as: bacteria, virus, fungi, parasites as well as preventing fat accumulation in body blood vessels, lowering blood pressure, lowering cholesterol level in the blood and destroying body organs cancer cells is done by inhaling vapors of fresh crushed or sliced garlic or vapors of synthetic allicin.

2. A method of claim 1 where the source of allicin is placed in a device in a manner that air flowing toward body respiratory system, absorbs allicin vapors and carry these vapors into the body of human or animal.

3. A method of claim 1 wherein the source of allicin is placed in a patient's mouth so when this patient breathes, allicin vapors are flowing into its respiratory system.

4. A method of claim 1 where allicin is created within a device so that air flowing toward the body mouth or nose absorbs allicin vapors thus introducing allicin into the body mouth or nose and to the respiratory system.

5. A method of claim 1, where synthetic allicin is generated by mixing alliinase and alliin.

6. A method of claim 1, where a source of allicin such as crashed or sliced garlic or synthetic allicin is placed in a device which allows airflow sucked by body lungs to absorb allicin and other vapors and carry them into the body mouth or nose.

7. A method of claim 1, where the airflow flows through at least one filter that stops garlic particles to be inhaled.

8. A method of claim 1, where the airflow flows through at least one filter that stops liquid droplets to be inhaled.

9. A method of claim 1, where a source of allicin such as crashed or sliced garlic or synthetic allicin is placed in a device which allows airflow, driven by powered fan, to absorb allicin vapors and other vapors and carry them into the body mouth or nose.

10. A method of claim 1, where the inhaled air, which contains allicin vapors, is being held in the lungs for few seconds and up to about 90 seconds.

11. A method of claim 1, where the allicin vapors is entering the body lungs thus allowing allicin molecules entering the body blood stream.

12. A method of claim 1, where the allicin vapors are filling the body lungs thus allowing allicin molecules destroying infection factors such as bacteria, virus, parasites and fungi who reside in the lung's bronchioles and alveoli.

13. A method of claim 1, where the allicin molecules passing the alveoli membranes and enter the body blood stream thus destroying infection factors such as bacteria, virus, parasites and fungi who reside in the body organs.

14. A method according to claim 1 for preventing and curing various lung diseases such as influenza, pneumonia, SARS, soar throat and asthma caused by fungi in the lungs.

15. A method of claim 1 where a patient breathes allicin vapors several times within few minutes, to be defined as inhaling session.

16. A method of claim 1, where said session is carried out for several times per day.

17. A device for implementing the method of claim 1, contains crushed or sliced fresh garlic has a convergent nozzle that accelerates inhaled air toward the garlic thus increasing the amount of absorbed allicin vapors.

18. A device for implementing the method of claim 1, contains at least one filter that stops garlic solid particles from leaving this device.

19. A device for implementing the method of claim 1, contains at least one filter that stops allicin droplets from leaving this device.

20. A device for implementing the method of claim 1, having a container where crushed garlic is placed.

21. A device for implementing the method of claim 1, having a container where at least one garlic clove is placed and crushed or sliced by the device.

22. A device for implementing the method of claim 1, having a electrical powered fan that drives air to flow and absorb allicin vapors and carry them toward a patient mouth or nose.

23. A device for implementing the method of claim 1, contains a mouth piece through which a patient breathes thus creating airflow that absorbs allicin vapors.

24. A method of claims 1, where an entire population is performing the inhaling allicin vapors session simultaneously according to mass media channels instructions like, radio television, newspapers, Internet, telephone and cellular phones.

25. A garlic scent inhaler device comprises of a pipe through which a patient inhales wherein preferably fresh crashed garlic particles or slices of garlic are placed at the pipe end opposite to the end placed in the patient mouth, thus when the patient breathes, air flowing over the crashed garlic absorbs garlic scent and this scent is being taken into the patient lungs.

26. A device according to claim 4 wherein said pipe cross section expands toward the patient mouth to slow inhaled air speed thus decreasing the aerodynamic force acting on garlic particles so they are not inhaled into the patient lungs.

27. A method according to claim 25 where vapors of allicin are generated in an inhaler, which contains allicin origin compounds in separated compartments.

28. A method according to claim 25 where vapors of allicin are generated in an inhaler, which contains alliinase and alliin in separated compartments.

29. An inhaler according to claim 26, where the inhaler contains a compartment of pressurized gas that push allicin droplet into patient mouth.
